(19) United States
(12) Patent Application Publication
(10) Pub. No.: US 2024/0197372 A1
(43) Pub. Date: Jun. 20, 2024

(54) APPARATUSES AND METHODS FOR TOE FIXATION PROSTHESIS

(71) Applicants: Daniel Preece, Sandy, UT (US); Benjamin Haslam, Hyrum, UT (US)

(72) Inventors: Daniel Preece, Sandy, UT (US); Benjamin Haslam, Hyrum, UT (US)

(21) Appl. No.: 18/535,952

(22) Filed: Dec. 11, 2023

Related U.S. Application Data
(60) Provisional application No. 63/387,447, filed on Dec. 14, 2022.

(51) Int. Cl. A61B 17/72; A61B 17/84; A61F 2/42

(52) U.S. Cl. CPC A61B 17/7291; A61B 17/7225; A61B 17/846; A61F 2/4225; A61F 2002/4228; A61F 2210/00

(57) ABSTRACT

Toe fixation prostheses and methods of implementation for the same are discussed herein. A toe fixation prosthesis to compress a first bone to a second bone may include and anchor member and a coupling member. The anchor member may include a first end to couple the anchor member to the first bone and a second end which includes a ramp. The coupling member may include one or more threads to couple to the ramp. The coupling member may be positioned transversally to a length of the second bone.

### RELATED APPLICATIONS

This application claims priority to U.S. Provisional Application No. 63/387,447, filed on Dec. 14, 2022 and titled, “Apparatuses and Methods for Toe Fixation Prosthesis,” which is hereby incorporated by reference in its entirety.

### TECHNICAL FIELD

The present application relates generally to toe fixation prostheses, and more specifically to methods and apparatus for using the same.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1A illustrates a perspective view of an anchor member, according to embodiments herein.

FIG. 1B illustrates a perspective view of an anchor member, according to embodiments herein.

FIG. 2 illustrates a perspective view of a coupling member, according to embodiments herein.

FIG. 3 illustrates a perspective view of an anchor member, according to embodiments herein.

FIG. 4 illustrates a perspective view of an anchor member, according to embodiments herein.

FIG. 5 illustrates a skeletal structure of a foot with an anchor member including a first end and a second end installed in an intermediate phalanx of a toe, according to embodiments herein.

FIG. 6 illustrates a skeletal structure of a foot with an anchor member installed in an intermediate phalanx of a toe and a coupling member installed in a distal phalanx of the toe, according to embodiments herein.

FIG. 7 illustrates a method according to embodiments disclosed herein.

### DETAILED DESCRIPTION

Medical procedures to correct for and/or alleviate symptoms of foot conditions such as hammer toe often aim to permanently stabilize the toe bones into a straightened or more natural position by surgically fusing the bones at the joints and in some cases using a metal pin to hold the bones in place until they fuse. Surgical failures can lead to complications such as non-union and/or mal-union of the corresponding toe bones. The apparatuses and methods for toe fixation prostheses described herein reduce the risk of post-operative non-union or mal-union of toe bones.

For example, as described herein, a toe fixation prosthesis for compressing a first bone to a second bone (e.g., a proximal phalanx and an intermediate phalanx, respectively) may include an anchor member and a coupling member. The anchor member may include one end to be coupled to the first bone and a second end having a ramp to be coupled to the coupling member. The coupling member may be positioned transversally to a length of the second bone. The anchor member may include one or more ramps with various relative configurations, which serve to compress the second bone to the first bone when one or more threads of the coupling member are coupled to the one or more ramps of the anchor member.

Accordingly, while specific examples recited herein may refer to fixation of a proximal phalanx to an intermediate phalanx of a toe, analogous concepts and apparatuses may be used in/with various other anatomical systems of the body, including one or more of the proximal, intermediate, or distal phalanges of the hand or foot, or otherwise at any joint between adjacent bones.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “proximal” and “distal” are used herein to refer to opposite directions along a patient's foot, with the proximal end of the foot being closer to the ankle and the distal end of the foot being at the end of the toes.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

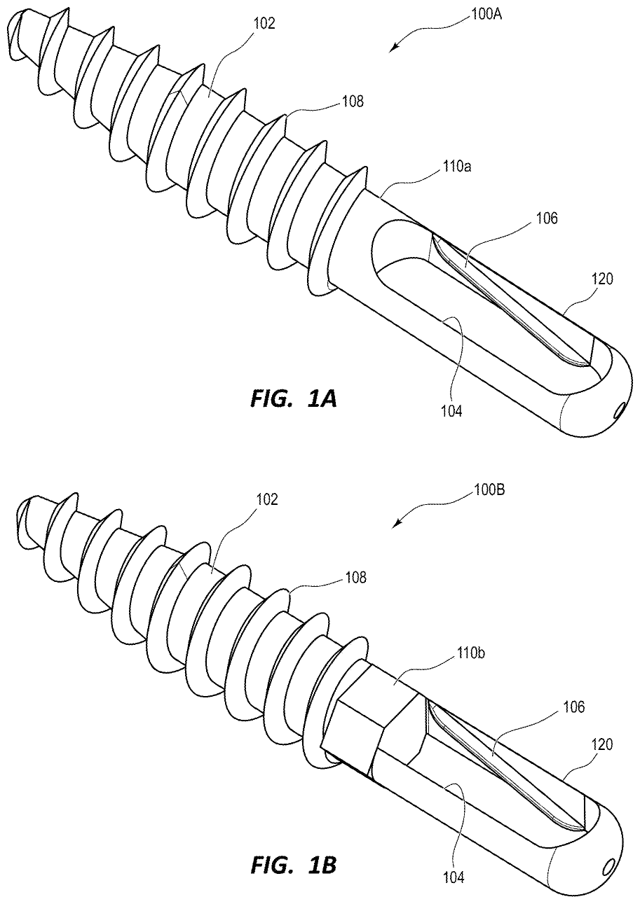

FIG. 1A illustrates a perspective view of an anchor member 100A, according to embodiments herein. In the illustrated embodiment, the anchor member 100A includes a first end 102 and a second end 120. The first end 102 can couple the anchor member 100A to a first bone. The second end 120 includes a loop 104 and a ramp 106. The first end 102 of the anchor member 100A can be referred to as a proximal end of the anchor member 100A, and is configured to couple the anchor member 100A to the first bone. Conversely, the second end 120 of the anchor member 100A can be referred to as a distal end.

The first end 102 of the anchor member 100A can be configured to couple to the first bone in various ways. In the depicted embodiment, the first end 102 has a helical thread such that it can be secured into the first bone (for example, in a direction along a length of the first bone). In some embodiments, the first end 102 may be secured into the first bone by being screwed into the first bone (e.g., as depicted, via threads 108). In other embodiments, the first end 102 may be anchored to the first bone using other various mechanisms, such as being anchored, pinned, clamped, etc. As used during certain therapies, the anchor member 100A is a structural member that couples to the first bone such that the longitudinal axes of the anchor member 100A and the first bone are coincident and or partially aligned.

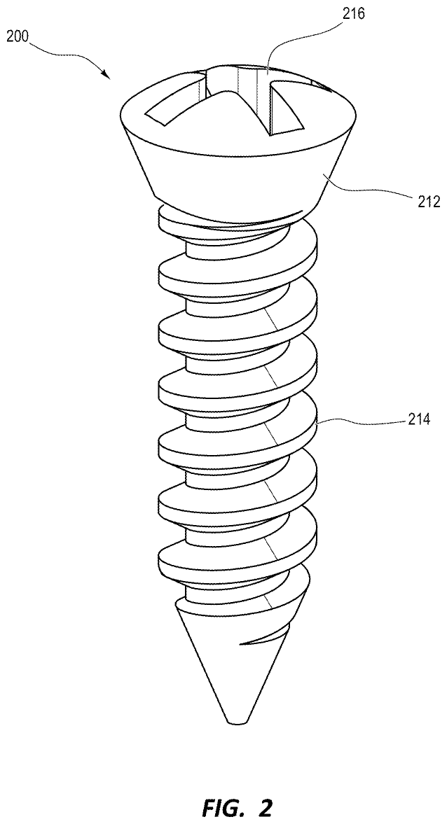

The second end 120 of the anchor member 100A is configured to be coupled with a coupling member, such as coupling member 200 of FIG. 2. As will be described in further detail in reference to FIG. 2, the coupling member can have one or more threads that couple to the ramp 106 to translate the coupling member along the longitudinal axis of the anchor member 100A (and thus the longitudinal of the first bone) upon a rotational motion of the coupling member along a transverse direction perpendicular to the longitudinal directions of the anchor member 100A and the first bone.

The ramp 106 is located within a loop 104 of the second end 120 of the anchor 100A. In particular, the ramp 106 is located and positioned within an inner perimeter of the loop 104. The ramp 106 is positioned such that it makes a non-zero angle with the longitudinal axes of the anchor member 100A and/or the first bone. The loop 104 is configured to receive the coupling member such as coupling member 200 of FIG. 2 and the ramp 106 is configured to couple to the coupling member 200 within the inner perimeter of the loop 104, as will be described in more detail with respect to FIG. 2.

In some embodiments, each of the components of the anchor member 100A are composed of titanium (Ti). For example, each of the components (the loop 104, the ramp 106, and the threads 108) of the anchor member 100A may be integral, or one or more of the components may be integral and the rest separate, and composed of Ti. The first end 102 and the second end 120 of the anchor member 100A are joined by a middle section 110a. In other embodiments, one or more of the components may be composed of Ti and the remaining components may be composed of some other suitable material. Similarly the coupling member (e.g., coupling member 200 of FIG. 2) may be composed of Ti or other suitable materials. Embodiments wherein one or more of the anchor member 100A and the coupling member (200 of FIG. 2) comprise titanium, stainless steel, polymers, and/or other materials are within the scope of this disclosure.

FIG. 1B illustrates a perspective view of an anchor member 100B, according to embodiments herein. The anchor member 100B is the same as or similar to the anchor member 100A of FIG. 1A except that the anchor member includes a hexagonal section 110b between the first end 102 and the second end 120. The hexagonal section 110b can be used to facilitate stabilization of the anchor member 100B either during installation of the anchor member 100B into the first bone (e.g., to provide rotational grasp) or during coupling of the coupling member 200 to the anchor member 100B (e.g., to provide hold and prevent rotation of the anchor member 100B.

As in the case of the anchor member 100A of FIG. 1A, the anchor member 100B may be a fully integral component or it may be constructed from one or more separate parts including the first end 102, the second end 120, and/or the hexagonal (middle section) 100b, and one or more of each of the parts is composed of Ti.

FIG. 2 illustrates a perspective view of a coupling member 200, according to embodiments herein. In some embodiments, the coupling member 200 is a screw that contains one or more set(s) of threads 214. The coupling member 200 is configured to be coupled with the ramp 106 (as depicted in FIGS. 1A-1B) through a second bone (as depicted in FIG. 6).

In some treatments, the coupling member 200 is positioned transversally through the second bone (which may be substantially aligned with the first bone), such that the second bone is located between a head 216 of the screw and the second end 120 of the anchor member 100.

The coupling member 200 serves to drive one end of the second bone into increasingly close contact with an adjacent end of the first bone upon rotation, thus reducing the gap between the first bone and the second bone. In other words, as the coupling member 200 is screwed and tightened, interaction between the threads 214 and the ramp 106, drives the second bone toward the first bone such that it may be compressed against the first bone.

In some embodiments, for example, as depicted, the drive of the coupling member 200 may be a Philips head (e.g., cross, plus, or the like). In other embodiments, the drive of the coupling member 200 may be flat, hexagonal, star, Allen, or other suitable configuration.

In many cases, it is desirable to minimize the gap between the first bone and the second bone in order to reduce the risk of non-union and/or mal-union of the bones during post-surgical healing of hammertoe or similar surgery.

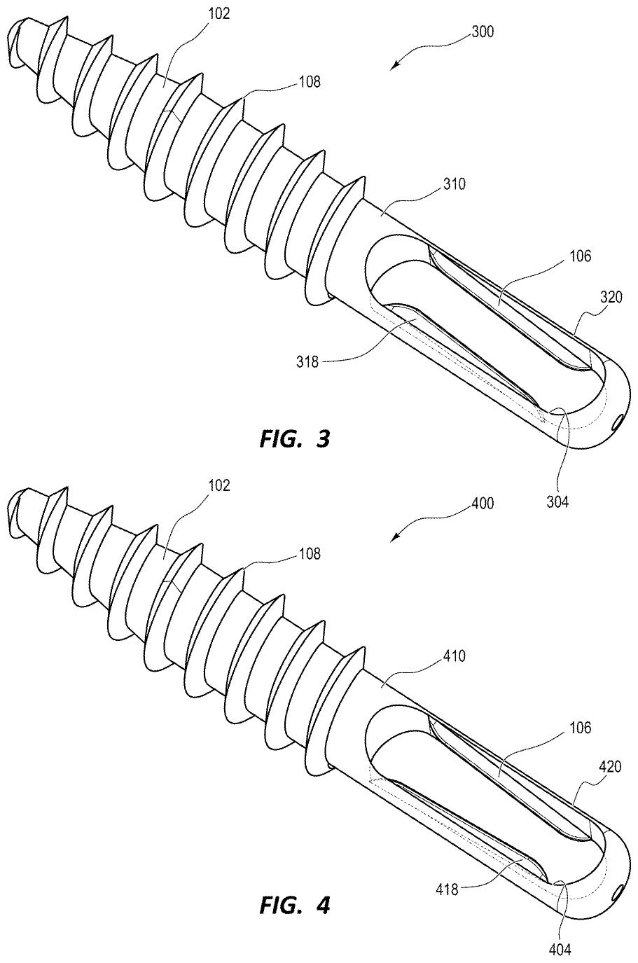

FIG. 3 illustrates a perspective view of an anchor member 300, according to embodiments herein. The anchor member 300 is the same as or similar to the anchor member 100A of FIG. 1A or the anchor member 100B of FIG. 1B, except that the anchor member 300 includes both the ramp 106 and a second ramp 318. As such, in some embodiments, a first end 102 and a second end 320 can be connected by a middle section 310 which may be cylindrical (as depicted) or hexagonal (as in FIG. 2).

The ramp 106 makes a non-zero angle with an axis that runs parallel to a longitudinal direction of the anchor member 300. The ramp 318 makes the same non-zero angle with the axis and is parallel to the ramp 106, such that the ramp 106 and the ramp 318 are parallel. The ramp 318 is located within the inner perimeter of the loop 304 opposite the ramp 106.

It is worth noting that although not depicted, in some other embodiments, the ramp 318 may make the same non-zero angle with the axis and is parallel to the ramp 106, but may be located within the inner perimeter of the loop 304 on the same side as the ramp 106 but shifted by a distance above or below the ramp 106. Furthermore, in some embodiments there may be additional similar ramps (e.g., third, fourth, fifth, etc.) located either on the same or opposite side of the loop 304 with respect to the ramp 106.

FIG. 4 illustrates a perspective view of an anchor member 400, according to embodiments herein. The anchor member 400 is the same as or similar to the anchor member 100A of FIG. 1A or the anchor member 100B of FIG. 1B, except that the anchor member 400 includes both the ramp 106 and a second ramp 418. As such, in some embodiments, a first end 102 and a second end 420 can be connected by a middle section 410 which may be cylindrical (as depicted) or hexagonal (as in FIG. 2).

The ramp 106 makes a first non-zero angle with an axis that runs parallel to a longitudinal direction of the anchor member 400. The ramp 418 makes a second angle with the axis. In some embodiments, the second angle is a negative fraction of the first non-zero angle. The second angle can be non-zero or the second angle can be zero. The ramp 418 is located within the inner perimeter of the loop 404 opposite the ramp 106.

It is worth noting that although not depicted, in some other embodiments, there may be additional ramps similar to ramp 106 or ramp 418 (e.g., third, fourth, fifth, etc.) but shifted to be located either above or below the respective similar ramp.

Ramps as described herein (including 106, 318, and 418) may comprise a flange or protrusion that extends from an inner surface of the loop (104, 304, 404). Additionally or alternatively, ramps may comprise shoulders, edges, ridges, recesses, channel, or other surfaces features on an inner surface of the loop. Continuous and discontinuous ramps are within the scope of this disclosure. Additionally, combinations of multiple ramps, on one or both sides of a loop, in any combination of angles are also within the scope of this disclosure.

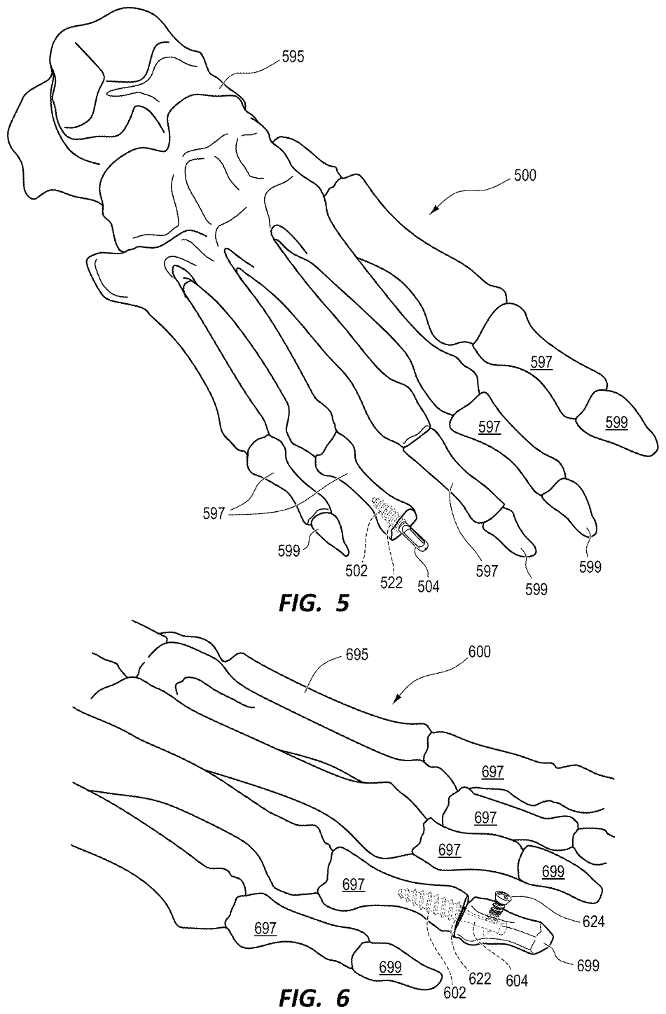

FIG. 5 illustrates a skeletal structure 595 of a foot 500 with an anchor member 522 including a first end 502 and a second end 504 installed in an intermediate phalanx of a toe, according to embodiments herein. The anchor member 522 may the same as or similar to any of the anchor members 100A, 100B, 300, or 400 of FIGS. 1A-1B, or FIGS. 3-4. As illustrated, the first end 502 of the anchor member 522 is coupled to an intermediate phalanx 597 of a toe. The second end 520 of the anchor member 522 protrudes from the intermediate phalanx 597 such that it can be coupled to a separate coupling member, such as the coupling member 200 of FIG. 2. As shown in FIG. 5, the anchor member 522 is secured such that its longitudinal axis is parallel and coincident with a longitudinal axis of the intermediate phalanx 597.

By coupling the second end 520 to the coupling member, which is disposed transversally to a length of a corresponding distal phalanx, a gap between intermediate phalanx and the distal phalanx can be minimized, thus reducing the risk of non-union and/or mal-union of the intermediate and distal phalanges following a surgical toe fixation procedure.

FIG. 6 illustrates a skeletal structure 695 of a foot 600 with an anchor member 622 installed in an intermediate phalanx 697 of a toe and a coupling member 624 installed in a distal phalanx 699 of the toe, according to embodiments herein. The anchor member 622 may be the same as or similar to the anchor members 100A and 100B of FIGS. 1A-1B, 300 of FIG. 3, 400 of FIG. 4, or 522 of FIG. 5. The coupling member 624 may be the same as or similar to the coupling member 200 of FIG. 2.

As illustrated in FIG. 6, the coupling member 624 can be a screw which is disposed through the distal phalanx 699 in a direction which is substantially transverse (e.g., perpendicular) to a longitudinal direction (e.g., along a length) of the distal phalanx 699. Upon a rotational motion of the coupling member 624 transverse to a longitudinal axis of the coupling member 624, the coupling member 624 can be translated along the longitudinal axis. In other words, by tightening the coupling member 624 (e.g., a screw), the coupling member 624 and the distal phalanx 699 are moved closer by way of one or more of the ramps 106, 318, or 418 of FIGS. 1A-1B, 3, 4 to the intermediate phalanx 697 in which the anchor member 622 is fixed.

As the coupling member 624 is rotated, threads on the coupling member (for example, threads 214 of FIG. 2) interact with ramps (such as 106, 318, 418) on the anchor member 622 drawing the coupling member 624 (and the distal phalanx 699) proximally and compressing the joint between the distal phalanx 699 and the intermediate phalanx 697.

In some embodiments, the coupling member 624 can be tightened via a clockwise rotational motion as viewed from above the head 212 of the coupling member 624. In other embodiments, the threads (e.g., threads 214) may be reversed and the coupling member 624 can be tightened via a counter-clockwise rotational motion as viewed from above the head 212 of the coupling member 624. In some embodiments, one or more of the ramps 106, 318, or 418 may be serrated, and/or comprise bumps or other features, such that the possibility of the coupling member 624 becoming accidentally loosened is minimized.

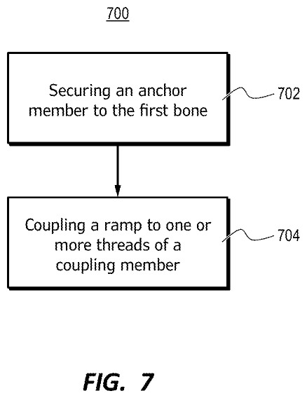

FIG. 7 illustrates a method 700 according to embodiments disclosed herein. The method 700 for compressing a first bone to a second bone includes securing an anchor member to the first bone (block 702) and coupling a ramp to one or more threads of a coupling member (block 704). The coupling member can be disposed transversally to a length of the second bone. The anchor member can include a first end and a second end. The first end can secure the anchor member to the first bone. The second end can include a loop and a (first) ramp positioned within an inner perimeter of the loop. The ramp can be coupled to the one or more threads of the coupling member.

In some embodiments, the method 700 further includes translating the coupling member along a longitudinal axis of the anchor member by rotating the coupling member transversally with respect to the longitudinal axis.

In some embodiments, the first bone refers to a proximal phalanx of a toe and the second bone refers to an intermediate phalanx of the corresponding toe. In other embodiments, the first bone and the second bone can refer to one or more of a distal, intermediate, or proximal phalanx of a toe bone.

In some embodiments, the anchor member can include a second ramp, also positioned within an inner perimeter of the loop and opposite to the first ramp. In some embodiments, the first ramp and the second ramp can both be oriented at a given angle with respect to the longitudinal axis of the anchor member, such that the first ramp and the second ramp are parallel. In other embodiments, the first ramp and the second ramp can be oriented at different angles with respect to the longitudinal axis. For example, the first ramp may be oriented at a first angle clockwise from the longitudinal axis and the second ramp may be oriented at a second angle counterclockwise from the longitudinal axis. In some embodiments the first angle and the second angle may have the same absolute value. In other embodiments, the first angle and the second angle may have different absolute values but opposite rotation directions with respect to the longitudinal axis. In each of the cases, the coupling member can couple to one or more of the first ramp and the second ramp.

In some embodiments, the anchor member can include a hexagonal portion between the first end and the second end. In some embodiments the anchor member can include a portion between the first end and the second end that has a cross section which is triangular, square, pentagonal, or the like.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents

What is claimed is:

1. A system for compressing a first bone to a second bone, the system comprising:
an anchor member comprising:
a first end configured to couple the anchor member to the first bone; and
a second end comprising a ramp; and
a coupling member comprising one or more threads to couple to the ramp, wherein the coupling member is disposed transversally to a length of the second bone.

2. The system of claim 1, wherein a rotational motion of the coupling member transverse to a longitudinal axis of the anchor member translates the coupling member along the longitudinal axis.

3. The system of claim 1, wherein the first bone is a proximal phalanx of a toe and the second bone is an intermediate phalanx of the toe.

4. The system of claim 1, wherein the anchor member further comprises a second ramp, the second ramp being disposed within an inner perimeter of a loop of the second end of the anchor member, and wherein the ramp is also disposed within the inner perimeter of the loop opposite the second ramp.

5. The system of claim 4, wherein the second ramp is parallel to the ramp.

6. The system of claim 4, wherein the ramp is oriented at a first angle with respect to a longitudinal direction of the anchor member and the second ramp is oriented at a negative fraction of the first angle with respect to the longitudinal direction.

7. The system of claim 1, wherein the second end of the anchor member comprises a loop, wherein the ramp is disposed within the loop, and wherein the coupling member couples to the ramp within an inner perimeter of the loop.

8. The system of claim 1, wherein the first end of the anchor member comprises a second coupling member.

9. The system of claim 1, wherein the anchor member further comprises a hexagonal section between the first end and the second end.

10. The system of claim 1, wherein the anchor member and the coupling member are titanium (Ti).

11. A method of compressing a first bone to a second bone, the method comprising:
securing an anchor member to the first bone, the anchor member comprising a first end to secure the anchor member to the first bone and a second end comprising a ramp; and
coupling the ramp to one or more threads of a coupling member, the coupling member being disposed transversally to a length of the second bone.

12. The method of claim 11, further comprising translating the coupling member along a longitudinal axis of the anchor member by rotating the coupling member transversally to the longitudinal axis.

13. The method of claim 11, wherein the first bone is a proximal phalanx of a toe and the second bone is an intermediate phalanx of the toe.

14. The method of claim 11, wherein the anchor member further comprises a second ramp, the second ramp being disposed within an inner perimeter of a loop of the second end of the anchor member, and wherein the ramp is also disposed within the inner perimeter of the loop opposite the second ramp.

15. The method of claim 14, wherein the coupling member couples to at least one of the ramp or the second ramp within the inner perimeter of the loop.

16. The method of claim 11, wherein the anchor member further comprises a hexagonal section between the first end and the second end.

17. An anchor member comprising:
a first end comprising a first coupling member;
a second end comprising a loop; and
a first ramp disposed within an inner parameter of the loop, wherein the first ramp is configured to couple to a second coupling member.

18. The anchor member of claim 17, further comprising a second ramp disposed within the inner parameter of the loop and opposite the first ramp, wherein the second ramp is configured to couple to the second coupling member.

19. The anchor member of claim 18, wherein the second ramp is parallel to the first ramp.

20. The anchor member of claim 18, wherein the first ramp is oriented at a first angle with respect to a longitudinal direction of the anchor member and the second ramp is oriented at a negative fraction of the first angle with respect to the longitudinal direction.
